(19) United States
(12) Patent Application Publication
(10) Pub. No.: US 2026/0145004 A1
(43) Pub. Date: May 28, 2026

(54) SLICE-SHAPED ABSORBER MASK FOR RADIATION THERAPY

(71) Applicant: Siemens Healthineers AG, Forchheim (DE)

(72) Inventor: Martin BRAEUER, Nuernberg (DE)

(73) Assignee: Siemens Healthineers AG, Forchheim (DE)

(21) Appl. No.: 19/396,905

(22) Filed: Nov. 21, 2025

(30) Foreign Application Priority Data
Nov. 22, 2024 (EP) 24214811.2
Dec. 23, 2024 (EP) 24222839.3
Mar. 25, 2025 (EP) 25166000.7

(51) Int. Cl. A61N 5/10; B33Y 10/00; B33Y 30/00; B33Y 80/00; G21K 1/10

(52) U.S. Cl. CPC A61N 5/10; G21K 1/10; A61N 2005/1095; B33Y 10/00; B33Y 30/00; B33Y 80/00

(57) ABSTRACT

An absorber mask for shaping a plurality of therapeutic x-rays includes a stack of slices. The stack of slices has a number of x-ray radiation-absorbing slices. The number of x-ray radiation-absorbing slices each have at least one planar layer. Neighboring layers of the stack of slices are applied to one another in a planar manner and in the stack direction by an additive manufacturing method. At least one x-ray radiation-transparent slice and/or at least one x-ray radiation-absorbing strip extending in the width direction is arranged between the number of x-ray radiation-absorbing slices.

### CROSS-REFERENCE TO RELATED APPLICATION(S)

The present application claims priority under 35 U.S.C. § 119 to European Patent Application No. 24214811.2, filed Nov. 22, 2024, European Patent Application No. 24222839.3, filed Dec. 23, 2024, and European Patent Application No. 25166000.7, filed Mar. 25, 2025, the entire contents of each of which are incorporated herein by reference.

### FIELD

One or more example embodiments of the present invention relate to the field of radiation therapy. For example, one or more example embodiments of the present invention relate to an absorber mask, to an arrangement for radiation therapy of a patient, to a method for manufacturing an absorber mask for shaping a plurality of therapeutic x-rays and to a system for additive manufacturing of an absorber mask for shaping a plurality of therapeutic x-rays.

### BACKGROUND

Radiation therapy is a widely-used method for therapeutic treatment of for example cancer and other illnesses, in which therapeutic radiation, especially therapeutic x-ray radiation, is used as ionizing radiation to prevent diseased cells from spreading.

According to the current prior art, absorber masks for shaping a plurality of therapeutic x-rays are manufactured mechanically from metal blocks or from sheets of metal. Due to its extremely high density and high screening effect with a given density, tungsten is a preferred material for these absorber masks. Tungsten is seldom used however since working it mechanically is extremely difficult and expensive. Other metals such as brass or iron are frequently used in the prior art, which can be worked far better mechanically, although they have a markedly lower screening effect. In a few cases the masks are also produced additively, by 3D printing methods for example. In such cases methods are employed in which metal powder is hardened, such as for example by selective laser melting (SLM).

The manufacturing of absorber masks with very fine structures is difficult and costly with conventional methods. Moreover, the materials used are often not optimally tailored to the specific requirements of different applications of radiation therapy. The lack of flexibility in the design of the absorber masks leads to compromises between dose maximization in the target region and protection of the surrounding tissue.

### SUMMARY

An underlying object of one or more example embodiments of the present invention is to specify an absorber mask, an arrangement for radiation therapy of a patient, a method for manufacturing an absorber mask for shaping a plurality of therapeutic x-rays and a system for additive manufacturing of an absorber mask for shaping a plurality of therapeutic x-rays, which can be manufactured in a manner which is improved, especially more flexible and preferably individual to the patient.

At least this object is achieved by the features of the independent claims. Advantageous embodiments are described in the dependent claims.

An embodiment of the inventive absorber mask for shaping a plurality of therapeutic x-rays, especially for a radiation therapy of a patient, has
a stack of slices,
wherein the stack of slices has a number of x-ray radiation-absorbing slices,
wherein the number of x-ray radiation-absorbing slices has at least one planar layer in each case,
wherein neighboring layers of the stack of slices are applied onto one another in a planar manner in the stack direction via an additive manufacturing method,
wherein at least one x-ray radiation-transparent slice is applied between the number of x-ray radiation-absorbing slices and/or at least one x-ray radiation-absorbing strip is arranged extending in the width direction.

An embodiment of an inventive method for manufacturing an absorber mask for shaping a plurality of therapeutic x-rays, especially for a radiation therapy of a patient, comprises the steps:
application via an additive manufacturing method of layers in a planar manner onto one another in the stack direction to form a stack of slices in such a way that
the stack of slices has a number of x-ray radiation-absorbing slices and
the stack of slices has at least one x-ray radiation-transparent slice and/or at least one x-ray radiation-absorbing strip extending in the width direction between the number of x-ray radiation-absorbing slices.

Embodiments of the present invention includes an innovative view of the direction of the additive production. This is because the layers of the (optional) x-ray radiation-transparent and x-ray radiation-absorbing slices are applied onto one another in a planar manner in the stack direction. The absorber mask is thus produced by many layers being applied above one another to form a stack of slices. Advantageously the absorber masks are thus produced by embodiments of an inventive system for additive manufacturing of an absorber mask being handled and employed so that thin layers of a minimal thickness are applied. To do this, the developing absorber mask is built up on a vertically movable plate, which is lowered and/or moved horizontally in the course of the production process. Such an arrangement is known from the area of additive manufacturing methods, for example from 3D printing, especially as a “core xy” arrangement. Basically it is conceivable, but not part of the present invention, to set the direction of production perpendicular to the upper side.

An absorber mask, regularly also referred to as an absorber raster or absorber grid, serves to align the therapeutic x-ray radiation onto the tissue of the patient to be treated. For this the absorber mask shapes a plurality of therapeutic x-rays, in that x-ray radiation-absorbing material shades out tissue to be protected. By contrast, openings between the x-ray radiation-absorbing material, which are filled with x-ray radiation-transparent material or can be free from material, starting from the therapeutic x-ray source, shape the plurality of therapeutic x-rays, which treat the tissue in the target region with a correspondingly high x-ray dose. These therapeutic x-rays the so-called “minibeams” or “microbeams” or also primary x-rays. Whether an x-ray photon is absorbed by the x-ray radiation-absorbing material or is transmitted as a therapeutic x-ray beam through the absorber mask especially depends on the angle of incidence of the trajectory on which the x-ray photon emitted for the therapeutic x-ray source is located in relation to the surface of the absorber mask. The surface of the absorber mask is especially on the upper side of the absorber mask.

The absorber mask is especially a passive apparatus, which can only shape therapeutic x-rays when it is correspondingly irradiated by a therapeutic x-ray source. The absorber mask itself does not create any therapeutic x-rays. The shaping of the plurality of therapeutic x-rays especially comprises an absorbing of such x-rays, which are not to strike the patient, and a transmission of those x-rays that, after passing through the absorber mask, are to have a therapeutic effect as therapeutic x-ray radiation in the target region in the tissue of the patient.

Therapeutic x-ray radiation is especially characterized by the x-ray photons having an energy of typically up to 80 keV, for example up to 140 keV, depending on the application case of the absorber mask. The X-ray photons typically have at least 40 keV, preferably at least 80 keV. The terms therapeutic x-ray radiation and x-ray radiation are used synonymously below.

In the present application the extents of a slice or a layer are designated as follows: In respect of the additive production method, in which material is typically applied layer by layer in a (main) direction of application, the length of the layer or of the slice relates to the extent in the direction of application. At right angles to this is the width of the layer or of the slice. The additive production method can be embodied to apply material at right angles to the direction of application in order to enlarge the width of the layer or of the slice. The length and the width lie especially in the plane in which the material is applied. The depth, also referred to as the thickness, of the layer or of the slice then refers to the extent perpendicular to this plane.

The extents of the absorber mask or of the stack of slices are further referred to as follows: the length of the absorber mask or of the stack of slices typically corresponds in this case to the length of the layer or of the slice. The width of the absorber mask or of the stack of slices is approximately produced by the sum of the thickness of all layers or of the slice. The width of the absorber mask or of the stack of slices thus essentially corresponds to the extent in the stack direction. The thickness of the absorber mask or of the stack of slices is essentially produced from the width of the layer or of the slice.

The stack of slices forms the basic structure of the absorber mask. A stack of slices refers to an arrangement of a number of layers lying one above another with different properties as regards the x-ray radiation, especially the x-ray radiation attenuation property. The x-ray radiation attenuation property defines the rate of absorption of x-ray radiation. The stack of slices is especially a layer stack with a number of layers of different properties. A slice especially has neighboring layers with the same x-ray radiation attenuation properties. Neighboring layers are especially those layers that are applied directly to one another. Neighboring layers with the same properties especially do not surround one or more layers with a different property. The at least one layer of an optional x-ray radiation-transparent slice and the at least one layer of the number of x-ray radiation-absorbing slices differ especially in their x-ray radiation attenuation property. The at least one layer of the x-ray radiation-transparent slice especially have a comparatively low x-ray radiation attenuation property and thereby a high x-ray radiation transmission property. The at least one layer of the number of x-ray radiation-absorbing slices especially has a comparatively high x-ray radiation attenuation property and thereby a low x-ray radiation transmission property.

The x-ray radiation-transparent slice typically comprises one or more neighboring x-ray radiation-transparent layers in each case and makes possible a high transmission of the primary x-ray radiation. The x-ray radiation-absorbing slices comprise at least one or more neighboring x-ray radiation-absorbing layers and absorb the x-ray radiation as effectively as possible.

An x-ray radiation-transparent layer can consist of materials with a low atomic number, for example plastics such as polyethylene, epoxy resin or polypropylene. These materials have a low x-ray radiation attenuation property and thus let a large part of the primary x-ray radiation pass through them. As an alternative or in addition materials with a low density such as foams and/or aerogels can also be used. The use of a carrier matrix made of a plastic, preferably polyethylene especially represents a good option, which is also frequently used in additive production. This material polyethylene is moreover suitable for use as an x-ray radiation-transparent material without a proportion of an x-ray radiation-absorbing material.

An x-ray radiation-absorbing layer typically consists of materials with a high atomic number and high density. Examples of this are metals such as especially lead or preferably tungsten, tantalum, rhenium, osmium, iridium, bismuth, platinum, thallium, mercury or gold. These materials absorb x-ray radiation effectively and can thus improve therapeutic x-ray radiation shaping. An x-ray radiation-absorbing layer made of tungsten is especially preferred.

A planar layer refers to a thin, extended surface of a material. The thickness of such a layer can lie in the range of a few micrometers to a few hundred micrometers, while the lateral extent is far greater and typically lies in the range of centimeters or more. The thickness of the layer thus describes the strength of the layer. A layer can be understood in this context as an individual, contiguous surface of the respective material, which is applied in a single production step. The thickness of such a layer can vary, depending on the material and desired properties used, between the layers and/or within the layer. For example, the thickness of the x-ray radiation-transparent layer can lie in the range of 20 to 2000 μm, while the thickness of the x-ray radiation-absorbing layers can lie in the range of 20 to 1500 μm, especially 20 to 500 μm. The thickness of a layer can vary depending on the material and desired properties used. The width of the layer can especially lie between 1 mm and 10 cm, advantageously between 2 mm and 3 cm. The length of the layer can especially lie between 1 cm and 100 cm, for example between 5 cm and 50 cm.

The additive manufacturing method makes possible the precise production of complex structures by applying layers of material, that is layer by layer. The additive manufacturing method is especially undertaken by the so-called “core xy” arrangement as part of embodiments of the inventive system. In the manufacturing of the absorber mask the individual layers are built up one after the other, wherein the layers for the x-ray radiation-absorbing slices are applied one after the other in the stack direction. In particular the at least one layer of one of the number of x-ray radiation-absorbing slices is applied to the uppermost or last of the layers of one of the number of x-ray radiation-absorbing slices already produced. When at least one x-ray radiation-transparent slice is arranged between the number of x-ray radiation-absorbing slices, the application is alternated between x-ray radiation-transparent material and x-ray radiation-absorbing material. The order for the production can also be reversed.

The additive manufacturing method can be method of building up slice by slice, in which the individual layers are applied one after the other. This method can make possible a precise control over the layer thicknesses and geometries. The layers can be applied by various techniques. One option can be the selective application of material by an application unit, for example with an application nozzle. The material can be applied here in liquid or paste or powder form, especially solid, or be subsequently hardened. Another option can be the selective hardening of a powder bed by introduction of energy, for example by a laser, for example by selective laser sintering, or of another energy source. As a further method the powder applied can also be hardened by a chemical process in which additional material is applied, for example by application, in particular spraying on, of a liquid, also called binder jetting. For the application of a layer it is conceivable for one or both of the material compositions to be applied as a liquid. In particular, x-ray radiation-transparent plastics, such as for example polyethylene, typically have a relatively low melting point of less than 300° C., while the x-ray radiation-absorbing materials, such as for example tungsten, can have comparatively high melt temperatures in the range of well over 1000° C., for example 3422° C. for tungsten.

An x-ray radiation-transparent slice can have one or more x-ray radiation-transparent layers. The number of x-ray radiation-absorbing slices can each have one or more x-ray radiation-absorbing layers. It is conceivable for the number of layers per slice to vary between the slices. The number of layers per slice can especially amount to 1, 2 or M>2. Preferably the neighboring layers of the stack of slices, i.e. the respective layers of various slices adjoining one another and the respective layers adjoining one another within a slice are applied to one another in a planar manner in the stack direction via the additive manufacturing method. The thickness of a slice cannot be below the thickness of the at least one layer of that slice. The thickness of a slice typically amounts to at least the sum of the thicknesses of all layers of the respective slice.

It is preferred for an x-ray radiation-absorbing slice to comprise exclusively x-ray radiation-absorbing layers and for an x-ray radiation-transparent slice to comprise exclusively x-ray radiation-transparent layers. It is conceivable, especially in respect of the choice of the production method, for one or more layers between an x-ray radiation-absorbing slice and an x-ray radiation-transparent slice to have a medium x-ray radiation attenuation property, for example on account of an unwanted contamination and/or for gradual setting of the x-ray radiation attenuation property. The amount of the medium x-ray radiation attenuation property especially lies between the lower x-ray radiation attenuation property of the x-ray radiation-transparent slice and the high x-ray radiation attenuation property of the x-ray radiation-absorbing slice.

As an alternative or in addition to the x-ray radiation-transparent slice, the absorber mask has the at least one x-ray radiation-absorbing strip extending in the width direction. When the absorber mask has additively applied x-ray radiation-transparent material and the x-ray radiation-absorbing strip within a layer, such a layer is by definition still an x-ray radiation-transparent layer. In other words, the interruption of the x-ray radiation-transparent material with the at least one x-ray radiation-absorbing strip does not change the purpose of this layer of basically not absorbing the x-rays completely, but of shaping the plurality of therapeutic x-rays. This applies equally when, within such a layer, no x-ray radiation-transparent material at all is arranged, but the at least one x-ray radiation-absorbing strip ensures as a type of spacer that the layers adjoining the at least one strip enclose an opening, especially a void, which is free of material.

In each layer only one x-ray radiation-absorbing strip or a number of x-ray radiation-absorbing strips can be arranged. The at least one x-ray radiation-absorbing strip covers a minimum 50%, for example more than 70% of the x-ray radiation-transparent layer. The at least one x-ray radiation-absorbing strip can be integrated into the otherwise x-ray radiation-transparent slices. This strip can consist of the same material as the x-ray radiation-absorbing slice or of another material with high x-ray absorption. The strip can be created for example by selective application of the absorbing material during the additive manufacturing process. The at least one x-ray radiation-absorbing strip can extend over the entire width direction of the layer or only over a part of it.

The purpose of these x-ray radiation-absorbing strips can consist of shaping the therapeutic x-rays in a further dimension. While the number of x-ray radiation-absorbing slices primarily reduce x-ray radiation in one direction, the at least one x-ray radiation-absorbing strip in the x-ray radiation-transparent layers x-ray can reduce radiation in a direction orthogonal thereto. The performance of the absorber mask can be influenced by this configuration in many respects. On the one hand an improved therapeutic x-ray radiation shaping in two dimensions can be achieved, which can lead to a higher quality for the radiation therapy. On the other hand the flexibility in the design of the absorber mask can be increased, since the density and/or arrangement of the at least one x-ray radiation-absorbing strip can be adapted to specific requirements. In particular, through this the absorber mask can take the form of a grid. The extent in the width direction as well as the length direction and the space between the at least one x-ray radiation-absorbing strip can be varied in order to obtain the desired absorption properties. For example the strips can have an extent in the length direction of the layer of 50 to 500 μm and be arranged at spaces of 1 to 10 mm. The thickness of the strip can correspond to the thickness of the layer into which it is integrated, or can differ from it.

The at least one x-ray radiation-absorbing strip can be integrated into or as an x-ray radiation-transparent slice by various methods. In the additive production method the x-ray radiation-absorbing material can be applied explicitly at desired positions, for example alternating with x-ray radiation-transparent material or alternating with one or more voids. As an alternative, prefabricated absorbing strips can be embedded into the transparent slices.

Upon application of the layers one above another additive production is especially undertaken in such a way that material that is applied in a layer below which a void is formed does not fill up the void but bridges it. In other words, the layers are preferably produced in such a way that the voids delimited by the additively produced strips remain free of material. The material of one layer especially does not fill up a void of another layer. A void especially continues to be free of material after the production of the absorber mask.

In summary, the variant described thus has the following advantages: This configuration can make possible an optimization of the absorber mask for specific applications in radiation therapy. By adaptation of the strip geometry and arrangement, the absorber mask can be adapted to various patients. Individual strips of x-ray radiation-absorbing material can be integrated in the layer consisting of x-ray radiation-transparent material, by controlling the system for additive manufacturing. Even if these strips, as a result of the process, can have a greater extent in the length direction than the layer thickness, especially due to the anisotropic minimal structure size, absorber masks can be produced in a grid form. Since the layer and number of the absorbing strips in the individual x-ray radiation-transparent layers can be predetermined in any given way, an absorber mask, the geometry of which is optimally adapted to the planned possible uses, is produced.

The absorber mask can have a plurality of slices overall. Typically the x-ray radiation-transparent slices and the number of x-ray radiation-absorbing slices alternate, especially regularly, preferably periodically, in the direction of alternation. The direction of alternation of the slices refers especially to the direction in which the x-ray radiation-transparent layer and the number of x-ray radiation-absorbing layers alternate. The direction of alternation of the slices especially runs in the direction in which the transmission rates of the layers and/or amounts of material alternate in respect of whole layers and merely just strips of x-ray radiation-absorbing material.

The stack direction of the layers especially relates to the stack direction of the manufacturing method, i.e. the direction in which the layers are applied in a planar manner one above another. The stack direction is typically at right angles to the plane of the planar layer. The stack direction runs, in accordance with one or more example embodiments of the present invention, essentially in parallel to the upper side or surface of the absorber mask. Thus the stack direction especially does not run at right angles to the upper side or surface of the absorber mask. In accordance with one or more example embodiments of the present invention, the direction of alternation of the slices corresponds to the stack direction of the layers.

An advantage of one or more example embodiments of the present invention thus lies in the fact that the critically thin structures are given by way of the layer thicknesses. It is especially advantageous that the neighboring layers of the stack of slices are or will be applied in the stack direction at the same time in the direction of alternation, since the surface of the individual layers can be created with a very broad application of material, for example greater than 100 μm or in the range of 0.1 mm to 1 mm, but especially thin or also thick in the thickness direction. This advantageously enables extremely thin especially x-ray radiation-transparent layers and thus x-ray radiation-transparent slices to be produced. As an alternative or in addition the production time of the individual layers is reduced compared to the conventional practice of building up the layers at right angles to the upper side of the absorber mask and thus at right angles to the direction of alternation. This is because, for a x-ray radiation-transparent slice, it has become necessary conventionally, especially in usual 3D printing methods, to divide this slice into 5 or more individual spatial points per layer, which makes production of the absorber mask with dimensions of for example 400 mm×400 mm or 300 mm×300 mm extremely expensive in the prior art.

Overall, the absorber mask described can thus offer a number of technical advantages. Through the use of the additive manufacturing method a very precise control of the layer thicknesses and geometries can be achieved. This can lead to an improved efficiency in radiation therapy. Furthermore, the use of alternative materials instead of lead can improve the environmental compatibility of the absorber mask. The possibility of producing complex structures can moreover make possible an optimization of radiation therapy for specific applications.

One form of embodiment makes provision for a minimum structure size within a layer to be greater than a minimum thickness of this layer. The minimum thickness of a layer can represent the smallest extent in the stack direction, which for example can be achieved for an individual layer in production terms. An example of this configuration can be a layer in which the minimum structure size within the layer amounts to 100 μm, while the minimum thickness of the layer can be 20 μm or 50 μm. At its heart, an anisotropic additive manufacturing method is described in this form of embodiment, in which the layers are very thin, for example a layer thickness is of the order of magnitude of 20 μm, while the material application in the two directions orthogonal to the stack direction has far coarser minimum structure sizes of greater than 20 μm, for example in the range of 50 μm to 1000 μm. The minimum structure size can relate to the smallest lateral dimension that can be manufactured within a layer of the absorber mask. This size can be determined by the resolution of the additive manufacturing method used. For example, in a 3D printing method, the minimum structure size can be influenced by the diameter of the cross section of the application nozzle of the application unit of the system for additive manufacturing or by the precision of the positioning of the application unit. As an alternative or in addition, the minimum structure size can depend on factors such as the viscosity of the material used, the surface tension and/or the hardening properties. For example in a stereolithography method the slice thickness can be precisely controlled by the penetration depth of the hardening light, while the lateral resolution can be restricted by the focus diameter of the laser. As an alternative, in a powder bed fusion method, the slice thickness can be determined by the height of the layer of powder applied, while the minimum structure size can depend on the grain size of the powder and the precision of the energy input.

The relationship between minimum structure size and minimum thickness can have various effects on the performance of the absorber mask. A larger minimum structure size compared to a minimum thickness can lead to an improved mechanical stability of the layer. This can be especially important for the x-ray radiation-absorbing slices, since these consist of more dense materials and can therefore be more susceptible to structural weaknesses. Moreover, this form of embodiment can make possible a layer thickness or slice thickness that is as small as possible. This can be of advantage for the optimization of the absorber mask.

One form of embodiment makes provision for a thickness of one of the number of x-ray radiation-absorbing slices to be greater than a thickness of the x-ray radiation-transparent slice or of the at least one x-ray radiation-absorbing strip. The thickness of one of the number of x-ray radiation-transparent slices can amount to less than 200 μm, especially less than 100 μm, preferably less than 50 μm, especially advantageously less than 25 μm. Preferably a thinner slice can minimize the absorption of the primary x-ray radiation, while at the same time an effective shaping of the therapeutic x-ray radiation is achieved. Examples of specific thicknesses within the said range can be 160 μm, 100 μm, 50 μm, 40 μm, 25 μm or 20 μm. A thickness of 100 μm can for example represent a good compromise between absorption capability and overall thickness of the absorber mask. The choice of the specific thickness can depend on various factors, such as for example the absorption material used and the specific requirements of the radiation therapy application.

One form of embodiment makes provision for one of the number of x-ray radiation-absorbing slices to have a maximum of one layer and/or wherein an x-ray radiation-transparent slice has a maximum of one layer. In the case the thickness of the one layer especially amounts to less than 200 μm, especially to less than 100 μm. This form of embodiment describes inter alia an absorber mask with a simplified slice structure, in which each of the slices-the x-ray radiation-transparent slice and the number of x-ray radiation-absorbing slices, consists of a maximum of one layer. The use of a maximum of one layer per slice can simplify the manufacturing process of the absorber mask. In an additive manufacturing method this can mean that each slice is applied in a single pass, which can reduce the production time and the material consumption. Moreover, this procedure can increase the precision of the slice thicknesses, since imprecisions that can arise from the multiple application of layers are void. Since each slice consists of a single layer, the composition and structure of the material within the slice can be more homogeneous, which can lead to a more even absorption or transmission of the x-ray radiation. A possible arrangement of the layers can look as follows: A single layer of the x-ray radiation-absorbing slice with a thickness of 100 μm, followed by a single layer of the x-ray radiation-transparent slice with a thickness of 20 μm, and finally a single layer of the x-ray radiation-absorbing slice with a thickness of 100 μm. By variation of the thickness and composition of the individual layers the mask can be optimized for specific applications in radiation therapy without increasing the complexity of the manufacturing process.

One form of embodiment makes provision for a cross section of a layer, especially a layer of the number of x-ray radiation-absorbing slices, to be embodied in the shape of a trapezoid in the width direction. A trapezoidal cross section can be understood in this connection as a geometrical shape in which the upper and lower edge of the layer run parallel to one another as well as parallel to the stack direction, but have different lengths. The side edges of the layer can run at an angle in this case and connect the upper edge with the lower edge. The width direction is perpendicular to the stack direction and also perpendicular to the upper side of the absorber mask. The trapezoidal shape is in particular a truncated wedge shape. It is conceivable for a cross section of a number of layers or all layers to be embodied in a trapezoidal shape in the width direction. The cross section of two or more layers can be different, especially at least have at least one different internal angle, that is to not cover the same area. For example the especially trapezoidal cross sections can be aligned differently, in particular slanted. The variation in thickness can be designed differently for various layers of the stack of slices. For example a different thickness variation can be used in an x-ray radiation-transparent layer from that used in an x-ray radiation-absorbing layer. This can make possible a precise tailoring of the absorption properties of the absorber mask.

The trapezoidal embodiment of the cross section of a layer can also be achieved by various production methods. In additive manufacturing methods for example the amount of material applied during the application of a layer can be explicitly varied in order to create the desired trapezoidal shape. In particular, a thickness of a layer of the stack of slices can be varied in the width direction via the additive manufacturing method. In a method with material applied as a liquid or a paste for example the amount of the material applied can be explicitly changed in the width direction of the layer. This can be carried out by adapting the flow of material, the speed of movement of the application unit or by a combination of the two parameters. In a powder-based additive manufacturing method, the variation in thickness can be achieved by selective hardening of different amounts of powder material in various areas of the layer. This can be realized by variation of the amount of energy that is used for hardening the powder, or by running over specific areas multiple times.

The inclination of the side edges of the trapeze can vary and be adapted to the specific requirements of the absorber mask. For example a tilt angle of between 0.001° and 45° can be chosen. A trapezoidal cross section of a layer can offer various potential advantages for the performance of the absorber mask. On the one hand this form can contribute to a focusing of the absorber mask. The oblique side edges can serve as a type of guide for the x-ray radiation and reduce undesired scattered x-ray radiation. It is significantly advantageous for the absorber mask to be focused on the target region in the tissue. This means in particular that slices in the center of the absorber mask are inclined at right angles to the surface, while slices with an increasing space from the center are inclined ever more in the direction of the target region. This can lead to an enhanced image quality in the radiation therapy. The trapezoidal embodiment of the cross section can contribute to optimization of the absorption properties of the absorber mask. The variation of the thickness within an in particular x-ray radiation-absorbing layer enables a gradual change in the absorption properties to be achieved. This can be especially useful for adapting the mask to specific radiation therapy modalities. As an alternative or in addition the trapezoidal form of a layer can contribute to reducing reflections and scatterings at the boundary surfaces between the layers. The use of a layer with trapezoidal cross section can in particular increase the flexibility in the design of the absorber mask. Variation of the trapeze shape in various layers enables three-dimensional structures within the absorber mask to be created, which are adapted to specific requirements of the radiation therapy.

One form of embodiment makes provision for the x-ray radiation-transparent slices to be embodied from a first material composition and the number of x-ray radiation-absorbing slices from a second material composition, wherein the first material composition and the second material composition are embodied differently from one another with regard to a proportion of a material. The first material composition and the second material composition can in particular be embodied differently from one another with regard to a proportion of exclusively one material or a number of materials. A proportion can lie between 0% and 100%. Proportions differ from one another by definition in this form of embodiment when, due to the difference, the x-ray radiation attenuation property of the first material composition differs from the x-ray radiation attenuation property of the second material composition in such a way that the first material composition is x-ray radiation-transparent and that the second material composition is x-ray radiation-absorbing. A material composition comprises a single material or a specific combination of materials, which together determine the properties of a slice. In particular the first material composition can just comprise one material, while the second material composition can comprise two or more materials. As soon as a material composition does not comprise a material of the other material composition, the two material compositions differ, since the proportion of the one material is equal to zero in the one material composition and not equal to zero in the other material composition. The material composition can comprise various materials, for example elements, compounds or structures in different proportions. The first material composition of the x-ray radiation-transparent slice can for example consist of lightweight materials with a low atomic number. Possible materials for this can be plastics such as polymers, polyethylene, epoxy resin, polypropylene or acrylic glass. In addition these materials can be provided with additives or fillers in order to achieve specific properties. The second material composition of the x-ray radiation-absorbing layer can comprise materials with a high atomic number. Such materials can be metals such as lead, tantalum, tungsten, rhenium, osmium, iridium, bismuth, platinum, thallium, mercury or gold, which can be embedded in the form of fine powders or nanoparticles into a carrier matrix, for example made of plastic.

One form of embodiment makes provision for the at least one x-ray radiation-transparent slice to be embodied from a first material composition, wherein the first material composition has hollow glass spheres and an aerogel. Hollow glass spheres, also known as micro hollow glass spheres or “microballoons” or glass microspheres, are microscopically small, hollow spheres of glass. These spheres typically have a diameter of a few micrometers, for example 80 μm, up to a few hundred micrometers and consist in particular of a thin glass shell, which encloses a gas volume or vacuum. Because of their structure hollow glass spheres have a very low density, typically in the range of 0.125 to 0.6 g/cm³ This low density, in combination with the properties of the glass makes hollow glass spheres an outstanding material for x-ray radiation-transparent slices. Technically, by mixing the hollow glass spheres with an epoxy resin or with a liquid, for example thermoplastic, a plastic material with very low density is able to be manufactured. With high concentrations of hollow glass spheres a type of foam with a density of below that of water (1.0 g/cm³) is produced. Advantageously the hollow glass spheres are employed for absorber mask production for reducing the density or absorption in such a way that: for example the hollow glass spheres are embedded in a thermoplastic or an epoxy resin in accordance with this form of embodiment. Overall the use of the hollow glass spheres makes possible a very marked reduction in undesired absorption of the primary x-ray radiation in an x-ray radiation-transparent slice. In particular, hollow glass spheres embedded in plastics make possible, through the very low density able to be achieved, the marked reduction in absorption of the primary x-ray radiation. This form of embodiment allows absorber masks to be produced significantly more effectively and reliably.

For the manufacture of slices with hollow glass spheres a method can be used in which the hollow glass spheres are embedded into a carrier matrix, in particular consisting of a plastic. This can be done by mixing the hollow glass spheres with the plastic, in particular a liquid plastic, and by subsequent hardening. As an alternative the hollow glass spheres can also be introduced into a powder bed and subsequently bound by sintering or melting.

As an alternative or in addition, hollow glass sphere-filled materials can also be supplemented or replaced by aerogels with an even lower density. Aerogels are highly porous solids with a low density. They consist for a large part of air (often over 95% of the volume) and have a network-like structure of nanoparticles connected to one another. Aerogels can be manufactured from various materials, wherein silica aerogels are the most widely known. The low density and the open structure likewise make aerogels an excellent material for x-ray radiation-transparent slices. These aerogels are typically available in far greater strengths of a few mm.

It is conceivable to employ aerogels in additive production, in particular in 3D printing. Aerogel slices in the range of 100 μm thickness in particular represent an addition or alternative to the use of hollow glass spheres.

In the use of aerogels a sol-gel method can be applied, in which first of all a gel is manufactured and the solvent is subsequently removed by overcritical drying, without destroying the pore structure. An alternative method is freeze drying, in which the solvent is removed by sublimation.

In both cases the additive manufacturing method can be adapted in order to make optimum use of the special properties of these materials.

The use of hollow glass spheres and/or aerogels in the first material composition can significantly improve the x-ray transparency of the corresponding slices, i.e. reduce the x-ray radiation attenuation property to the benefit of a higher x-ray radiation transmission property, which in turn can increase the efficiency of the absorber mask. The reason for this is that both materials have a high proportion of air or gas, which lets x-ray radiation pass through almost unhindered. At the same time these materials can positively influence the mechanical stability and other important properties of the slices. In particular the low density of these materials can contribute to a reduction in the overall weight of the absorber mask, which can be advantageous in specific applications. For example special application nozzles can be used, which make possible an even distribution of the hollow glass spheres or a controlled deposition of the aerogel.

One form of embodiment makes provision for the number of x-ray radiation-absorbing slices to be embodied from a second material composition, wherein the second material composition features lead, tantalum, tungsten, rhenium, osmium, iridium, bismuth, platinum, thallium, mercury or gold. Especially advantageously these elements, i.e. the said metals of the 6th period, are embedded in the carrier matrix described and employed in the an embodiment of the inventive system for additive production. Through this, almost complete freedom of the geometrical design of absorber masks preferably exists. The second material composition in particular forms a number of x-ray radiation-absorbing slices of the absorber mask. The use of the aforementioned materials with high atomic number and high density is decisive for an effective absorption of x-ray radiation. The said elements have these properties and can therefore be especially suitable for use in the x-ray radiation-absorbing slice.

Because of its high density of around 11.3 g/cm³ and its good absorption properties, lead can be used for x-ray radiation. Lead, as an element of the 6th period in the periodic system, has the highest relevant atomic number for x-ray radiation absorption. Because of their increasingly low atomic numbers the following stable elements of the 6th period in particular come into question as a substitute for lead, under certain conditions also thallium and mercury, despite their toxicity. In particular the preferred elements tantalum, tungsten, rhenium, osmium, iridium, bismuth, platinum or gold elements come in question as a substitute for lead. As well as the atomic number of the preferred elements, their density is very relevant. With the given material thickness for lead (for example 20 μm) the material thickness needed for the preferred elements is able to be calculated. Tantalum has a higher density of around 16.7 g/cm³ than lead. Tungsten, with a density of around 19.3 g/cm³ can offer an outstanding absorption capability for x-ray radiation. Rhenium, with a density of around 21.0 g/cm³ has a very high absorption capability. Osmium has the highest density of the said elements of around 22.6 g/cm³. Iridium, with a density of around 22.6 g/cm³ can likewise have a very high absorption capability. Platinum, with a density of around 21.5 g/cm³ can offer an excellent absorption capability for x-ray radiation. Gold, with a density of around 19.3 g/cm³ can offer a very good absorption capability. Mercury, with 13.6 g/cm³ and thallium with 11.7 g/cm³ have a higher density than lead, while the density of bismuth, with 9.8 g/cm³ is lower.

The result is that all the preferred elements mentioned above can be employed more advantageously than lead, since, with a simultaneous screening effect, the result is smaller material thicknesses. Of the said elements, tungsten is the one which comes into consideration for use above all because of its relative low price. The elements osmium, gold and platinum, because of their comparatively high prices, can specifically be excluded for commercial reasons for use in absorber masks.

These materials can be embedded into the absorber mask in various ways. One option can be the embedding of fine powders or nanoparticles of these materials into a carrier matrix or alloys with such materials.

The use of these materials in the second material composition can offer various advantages. On the one hand a high absorption efficiency can be achieved, which can lead to effective shaping of the plurality of therapeutic x-rays. On the other hand, through the choice of the material and the form of processing, the thickness of the absorbing layer can be optimized, which can lead to a reduction in the overall thickness of the absorber mask. What is more, the use of alternative materials to lead can enhance the environmental compatibility of the absorber mask.

With known methods many metals can be deposited chemically or electrochemically from an aqueous solution. In the actual choice of the element it is important that the element is able to be deposited industrially. Here in particular, despite its high price, gold is a good alternative, since corresponding methods of depositing gold are well established in the electrical engineering industry for refinement of contact surfaces. Only elements of the 6th group from rhenium to bismuth can be deposited electrochemically from an aqueous solution. Because of its chemical makeup, tungsten cannot be deposited from an aqueous solution. Rhenium comes into question, in accordance with one or more example embodiments of the present invention, for an electrolytic deposition and also has the lowest price of the metals mentioned above. However, with methods that have recently become known (see Dominik Höhlich et. al.: Simultaneous Electrodeposition of Silver and Tungsten from [EMIm]Cl:AlCl3 Ionic Liquids outside the Glove Box; Coatings 2020, 10(6), 553; https://doi.org/10.3390/coatings10060553, DOI: 10.7395/2020/Hoehlich and the patent DE 10 2014 118 593 A1) tungsten can be deposited together with nickel or silver from an ionic (non-aqueous) solution. In particular a corresponding deposition of tungsten or tungsten in conjunction with another metal is possible.

Tungsten comes into question as a relatively low-cost element. Since tantalum is also non-toxic as a material and it still has a slightly better x-ray radiation attenuation property than lead, it can also be provided as a material for the x-ray radiation-absorbing slice. In particular tungsten-rhenium alloys can be employed. The advantage of these alloys is that the mixture ratio of the two elements typically has no role to play in the x-ray radiation attenuation property, since this is almost identical for both elements. Alloys consisting of tungsten with for example copper, nickel and/or iron that can be easily shaped also represent a further option. Since technically these alloys are offered with typical tungsten content of 90% to 95%, the lower x-ray radiation attenuation property of the added lightweight elements, for example copper has around 9% of the absorption capability by volume of lead, typically only leads to a slight increase in the thickness of x-ray radiation-absorbing slices needed.

As an alternative to the alloys with the preferred elements, mixtures of powders of the preferred elements with other binder substances can be used for manufacturing the x-ray radiation-absorbing slices. As binders or matrix material, both metals such as in particular silver or tin come into question, and also plastics. The embedding of tungsten powder into tin is known from DE 60 2004 000 309 T2. As an alternative, plastics in particular are suitable as a material for embedding of tungsten powder. Commercial materials with a tungsten powder content of over 90% in a carrier matrix made of polyethylene are available. These materials have densities of up to 15 g/cm³.

One form of embodiment makes provision for the first material composition to have a plastic matrix and for the second material composition to have the same plastic matrix. The plastic matrix is in particular a carrier matrix made of plastic. The two material compositions have the same plastic matrix, which does not mean that the two material compositions are identical. The two material compositions can additionally each have a material that is not contained in the other respective material composition. It is conceivable for the first material composition to consist of the plastic matrix and for the second material composition to have a proportion greater than zero of an x-ray radiation-absorbing metal, such as for example the aforementioned materials, which is embedded into the same plastic matrix. The plastic matrix refers to a polymer material, which serves as a basic structure or binding mechanism for other materials. In the absorber mask of this form of embodiment this carrier matrix is used both as an x-ray radiation-transparent slice and also in the number of x-ray radiation-absorbing slices. For an x-ray radiation-transparent slice in particular the plastic matrix can consist of a material with a low atomic number. These materials have a low absorption of x-ray radiation and can therefore make possible a high transmission of the primary x-ray radiation. In a number of x-ray radiation-absorbing slices the same plastic matrix is used in this form of embodiment, but especially with additions of materials with a high atomic number however. These additions can in particular be embedded into the matrix in the form of fine powders or nanoparticles. Preferably tungsten or tantalum powder can be worked into the plastic matrix in order to increase the x-ray absorption. The use of the same plastic matrix in both material compositions can offer a number of advantages. First of all an improved adhesion between the slices can be achieved, since the chemical compatibility between the materials is increased. This can lead to an improved structural integrity of the overall absorber mask. Furthermore, the use of the same matrix can simplify the manufacturing of the absorber mask. With additive production methods for example the same basic material can be used for all slices, wherein only one addition or the additions is or are varied for the x-ray radiation-absorbing slice. This can design the production process more efficiently and avoid possible compatibility problems between various matrix materials. Moreover the use of a uniform matrix can lead to a more even thermal extent of the various slices. This can reduce thermal stresses and possible deformations of the absorber mask with changes in temperature. The choice of the specific plastic matrix can depend on various factors, such as for example the desired mechanical stability, the workability in the additive production process and the compatibility with the additional materials used for the absorbing slice.

As an alternative it is conceivable for the first material composition and the second material composition to have different plastic matrices. In particular the two plastic matrices differ in the type of the respective plastic, for example PET and PP.

Ideally the machine has at least two application nozzles, with which different materials are applied: An x-ray radiation-absorbing material that is as strong as possible with one of the two application nozzles and a material that is as x-ray radiation-transparent as possible with the other of the two application nozzles. Basically it is conceivable that either x-ray radiation-absorbing material or x-ray radiation-transparent material is applied with the two application nozzles simultaneously, one after another and alternating layer by layer.

The absorber mask arises from an alternating construction of layers of the two materials. A meltable matrix, which is filled with a high proportion of fine tungsten powder or other powders of elements of the 6th period of the periodic system of elements, preferably comes into question as a heavily x-ray radiation-absorbing material. X-ray radiation-absorbing means, in particular, as x-ray-proof as possible by comparison with x-ray radiation-transparent.

One form of embodiment makes provision for a space within a layer between x-ray radiation-absorbing strips to amount to less than or equal to 10 mm, preferably to less than or equal to 1 mm. The space relates to the longitudinal direction of the layer. As an alternative or in addition, a space within a layer between x-ray radiation-absorbing strips can amount to greater than or equal to 0.01 mm, preferably greater than or equal to 0.1 mm. In particular a ratio within a layer between a length of an x-ray radiation-absorbing strip and a neighboring space can amount to greater than or equal to 1, preferably to greater than or equal to 2. The space between two x-ray radiation-absorbing strips can be filled with x-ray radiation-transparent material or be free of material.

One form of embodiment makes provision for x-ray radiation-absorbing strips of neighboring layers to be combined to form a regular structure. A regular structure refers in this context to a geometrical shape with N corners and N sides, which is formed by the arrangement of the x-ray radiation-absorbing strips in neighboring layers. In particular the regular structure is a rectangle or square. Further examples of regular structures can be triangles (N=3), pentagons (N=5), hexagons (N=6) or more complex polygons with a higher number of corners. An absorber mask of this form of embodiment has a grid shape. The specific choice of N can depend on the desired absorption properties and the intended use of the absorber mask. The formation of these regular structures can be achieved by a precise arrangement of the x-ray radiation-absorbing strips in consecutive layers. For example, in a first layer, strips can be arranged at a specific position in relation to the longitudinal extent, while in the layer lying above, the strip is arranged at an offset position or at the same position. The overlaying of these strips enables the regular structures to be produced.

This form of embodiment can influence the performance of the absorber mask in multiple respects. On the one hand an improved shaping of the plurality of therapeutic x-rays in various directions can be achieved, since the regular structures can absorb x-rays from different angles. On the other hand this arrangement can lead to a more even distribution of the absorption over the surface of the absorber mask.

The manufacturing of such an absorber mask with regular structures can be realized by additive production method. In these methods the x-ray radiation-absorbing strips can be precisely positioned in each layer in order to create the desired regular structures. These methods can make possible a high precision in the creation of the regular structures. The size of the N-cornered structures can be varied in order to adapt the absorption properties of the absorber mask to specific requirements. Smaller structures can make possible a finer control of the shaped therapeutic x-rays, while larger structures are possibly easier to produce and overall bring about less x-ray radiation absorption.

One form of embodiment makes provision for the stack of slices to be produced depending on a contour of a patient in such a way that one side of the absorber mask essentially features the negative of the contour of the patient. The side that features the negative is especially a contoured side of the absorber mask. The contoured side featuring the negative means especially that the surface of the side is contoured so that it matches the contour of the patient. The contoured side is for example the upper side of the absorber mask. The contour of the patient is especially predetermined by a surface, for example the skin, of the patient. For example an image can show the contour of the patient, which will be taken into account in the production of the stack of slices. The image can be a photograph or a 3D image or a 3D pattern or another three-dimensional representation. The contour of the patient can be available in pixels or in vector or polygon data. The absorber mask typically has an extent, which is small by comparison with the patient. The absorber mask is usually restricted to the size in which the target region lies in tissue. Thus the stack of slices does not depend on the contour of the entire patient, but only on a usually small proportion of the patient. The contoured side of the absorber mask is preferably embodied for an exact fit on the contour of the patient. For example the target region can lie in the tissue of the upper arm of a patient. In this case the contoured side of the stack of slices is formed in such a way that the absorber mask can fit exactly onto the upper arm. An exact fit especially means without gaps. This form of embodiment is especially advantageous because the absorber mask is produced for an individual patient. This preferably enables the quality of the radiation therapy to be enhanced. Furthermore the patient-individual absorber mask can make an expensive apparatus for alignment and/or the fixing of the absorber mask relative to the patient superfluous.

An embodiment of an inventive arrangement for a radiation therapy of a patient has
an inventive absorber mask,
a therapeutic x-ray source and
a therapy area,
wherein the absorber mask is arranged between the therapy area and the therapeutic x-ray source and is aligned so that the stack direction of the stack of slices is essentially perpendicular to the x-ray radiation of the therapeutic x-ray source.

An x-ray source can be an apparatus that creates the x-ray radiation. The x-ray source can be embodied to create x-ray radiation with specific energy or a particular energy spectrum. The x-ray source can especially be an x-ray emitter or an accelerator system. The x-ray emitter comprises in particular an evacuated housing, in which in particular an anode and a cathode are arranged. The cathode comprises in particular an electron emitter, by which free electrons can be accelerated in the direction of the anode via a high voltage or high frequency pulses. When the accelerated electrons strike the anode the x-ray radiation in particular is generated. The electron emitter can especially be a cold, for example a field effect emitter, or a thermionic, for example a helical or sheet metal emitter. The method described is not however restricted to the exemplary method and apparatus for x-ray radiation given however. X-ray sources based on “inverse Compton scatter” are also known, for example. In this case x-ray radiation is created by the interaction of high-energy electrons and very intensive laser beams. Furthermore, there are also x-ray sources known in which anodes made of liquid or powdered metals are employed.

A therapy area can be a space or volume in which the patient to be treated can be placed. The therapy area is especially a treatment area. The arrangement of the components can be designed so that the x-ray source emits x-ray radiation, which in the therapy area strikes the patient for radiation therapy. The absorber mask can be positioned between the therapy area and the x-ray source in order to shape the plurality of therapeutic x-rays, especially for the radiation therapy of the patient.

Since an embodiment of the inventive arrangement has an embodiment of the inventive absorber mask, it shares the aforementioned advantages. The alignment of the absorber mask with the stack direction of the stack of slices essentially at right angles to the x-ray radiation can serve to optimize the quality of the radiation therapy. The arrangement for a radiation therapy described can lead to a minimization of the absorption of the primary x-ray radiation.

Embodiments of the inventive arrangement can in particular be suitable for a radiation therapy. An application of radiation therapy is especially an irradiation with very inhomogeneous fields, for example “spatially fractionated radio therapy”.

A radio therapy system can comprise an embodiment of the inventive arrangement and also for example at least one control computer, which can control the x-ray source for radiation therapy. The radio therapy system can especially be embodied for radiation therapy.

An embodiment of an inventive system for additive manufacturing of an absorber mask for shaping a plurality of therapeutic x-rays, especially for a radiation therapy of a patient, has an application unit, which is embodied to carry out an embodiment of the inventive manufacturing method.

An application unit can be an apparatus which is embodied to apply material in slices in order to create a three-dimensional structure. The application unit can comprise various components, such as for example one or more application nozzles, extruders or other mechanisms for precise application of material. The application unit can be embodied to process and to apply various materials. These materials can comprise x-ray radiation-transparent materials of the first material composition for the x-ray radiation-transparent slice and x-ray radiation-absorbing materials of the second material composition for the number of x-ray radiation-absorbing slices. The application unit can have mechanisms available to switch between various materials or to apply a number of materials at the same time. The system can be configured so that the application unit applies layers in a planar manner onto one another in the stack direction to form a stack of slices. In such cases the system can ensure that the stack of slices has the number of x-ray radiation-absorbing slices and between the number of x-ray radiation-absorbing slices at least one x-ray radiation-transparent slice and/or at least one x-ray radiation-absorbing strip extending in the width direction, wherein each of these slices has at least one planar layer. The system can comprise control mechanisms, which make it possible to precisely control the thickness of the layers applied. This can be especially important for the manufacturing of absorber masks with very thin absorbing slices. The system can have positioning systems available, which make possible a precise movement of the application unit in three dimensions. This can make possible an exact control over the geometry and structure of the absorber mask manufactured. The system can also comprise systems for monitoring and quality control of the manufacturing process. These can for example include sensors for measuring the thickness of the slice or for checking the material composition. Such a system for additive manufacturing of an absorber mask can make possible a high flexibility in manufacturing. The geometry and composition of the absorber mask can be easily adapted in order to fulfil various requirements in radiation therapy. Moreover, the additive production can make possible the manufacturing of complex internal structures that would be difficult to realize with conventional production methods. In addition the system can be embodied to vary the thickness of a layer of the stack of slices in the width direction. This can be achieved by precise control of the application of material during the additive manufacturing process, for example by adapting the material flow and/or the speed of movement of the application unit.

One form of embodiment makes provision for the application unit to have at least one application nozzle with an oval or rectangular cross section. The cross section of the application nozzle relates to the opening of the application nozzle from which the material emerges. The cross sections of a number of application nozzles can differ in form and/or in size. An application nozzle with an oval or rectangular cross section can offer a number of advantages for the manufacturing process and for the resulting structure of the absorber mask. By contrast with a conventional round nozzle, an oval or rectangular nozzle can apply a wider material track in one single pass. This can reduce the production time and increase the efficiency of the manufacturing process.

The oval or rectangular cross section of the application nozzle can make it possible to apply material slices with a controlled width and thickness. The form of the nozzle can be chosen so that the material track applied corresponds to the desired geometry of the absorber mask. By way of example, a rectangular nozzle with a side ratio of 10:1 can apply a material track that is 10 times as wide as it is thick.

The use of an oval or rectangular application nozzle can also contribute to improving the surface quality of the absorber mask manufactured. The wider application of material enables surface unevenness that can arise from the layer-by-layer application to be reduced. This can lead to a smoother surface of the absorber mask, which can be advantageous for the performance in radiation therapy. Moreover, the form of the application nozzle can be adapted to the specific requirements of the various slices of the absorber mask. For the x-ray radiation-transparent layers for example, a wider application nozzle can be used in order to make possible a rapid coverage of large surfaces. A narrower nozzle can be employed for the x-ray radiation-absorbing layers in order to guarantee a more precise control over the slice thickness.

The oval or rectangular form of the application nozzle can also contribute to optimizing the internal structure of the absorber mask. By explicitly aligning the application nozzle during the application, material chains can be created that are oriented in a specific direction. This can be used in order to control the absorption properties of the mask in various directions. A system for additive manufacturing of an absorber mask with an application unit, which has at least one application nozzle with an oval or rectangular cross section can make possible an improved control of the manufacturing process and the resulting mask structure. This can contribute to an optimization of the performance of the absorber mask in radiation therapy, especially through the focusing of the absorber mask.

One form of embodiment makes provision for the system to have a tilt apparatus and for the application unit and also the tilt apparatus to be arranged in such a way that, via the tilt apparatus, a tilt angle of the application unit is able to be changed relative to a layer already applied. The tilt apparatus can be a mechanical system that makes it possible to incline the application unit about one or more axes. This tilt apparatus can for example consist of joints, hinges or pivot apparatuses, which allow a precise control of the angle of the application unit. The arrangement of the application unit and the tilt apparatus can be designed so that a change in the tilt angle of the application unit relative to a layer that has already been applied is possible. This means that the application unit can be positioned at various angles to the surface of the last layer of material applied. Through the tilt axis of the tilt apparatus the absorber mask arising for example on a vertically movable plate can be tiltable, via the tilt apparatus, with different tilt angles relative to the surface in which the application unit is moving. As an alternative or in addition, the application unit can be tiltable against the horizontal by the tilt apparatus. The change of the tilt angle can be undertaken by a manual setting or by an automated control. An automated control can for example be realized by servo motors or actuator drives that are connected to a control unit. This control unit can adapt the tilt angle based on pre-programmed parameters or in real time during the production process. The variable tilt angle of the application unit can make it possible to manufacture focused absorber masks. In a focused absorber mask the absorbing structures are aligned so that they are aligned to a specific point, typically the x-ray source. This can be achieved by a gradual change in the angle of the applied layers.

The use of the tilt apparatus enables the angle of each layer applied to be precisely controlled. For example, the tilt angle can be set so that the layers in the center of the mask are applied parallel to the surface, while the layers at the edge are increasingly tilted inwards. This can lead to an improved efficiency of the absorber mask, since the absorbing structures are aligned optimally to the x-ray source. Moreover, the tilt apparatus can contribute to improving the surface quality of the manufactured absorber mask. Adaptation of the tilt angle enables the application of material to be controlled so that overhangs or undercuts are avoided, which can lead to a smoother surface and an improved structural integrity of the mask. The use of a tilt apparatus in the system for additive manufacturing of an absorber mask can make possible an increased flexibility and precision in the manufacturing process. This can contribute to an improved performance of the absorber mask in radiation therapy, in that the geometry and alignment of the absorbing structures can be adapted optimally to the specific requirements of the respective application.

An embodiment of inventive use of a system with an application unit for additive manufacturing of an absorber mask is likewise part of the present invention. This use relates to the use of the system described above or of other systems with an application unit for additive manufacturing for the method for manufacturing an embodiment of the inventive absorber mask. Each system with such an application unit can typically be used for building up the various layers of the absorber mask precisely and efficiently.

Features, advantages or alternative forms of embodiment mentioned in the description of the apparatus are likewise to be transferred to the method and vice versa. In other words claims for the method can be further developed with features of the apparatus and vice versa. In particular, embodiments of the inventive apparatus can be used in the method.

### BRIEF DESCRIPTION OF THE DRAWINGS

Exemplary embodiments of the present invention are shown in the drawings and are described in greater detail below. The same reference numbers are used for the same features in different figures.

In the Figures:

FIG. 1 shows a schematic diagram of a first variant of an inventive absorber mask;

FIG. 2 shows a perspective view of a second variant of an inventive absorber mask;

FIG. 3 shows a perspective view of a third variant of an inventive absorber mask;

FIG. 4 shows a schematic diagram of a first exemplary embodiment of the first variant of the inventive absorber mask;

FIG. 5 shows a schematic diagram of a second exemplary embodiment of the first variant of the inventive absorber mask;

FIG. 6 shows a schematic diagram of an inventive arrangement for radiation therapy;

FIG. 7 shows a schematic diagram of a first exemplary embodiment of the arrangement for radiation therapy;

FIG. 8 shows a flow diagram of an inventive method;

FIG. 9 shows a perspective view of an inventive system for additive manufacturing of an absorber mask;

FIG. 10 shows a schematic side view of a first exemplary embodiment of the system and

FIG. 11 shows a schematic side view of a second exemplary embodiment of the system.

### DETAILED DESCRIPTION

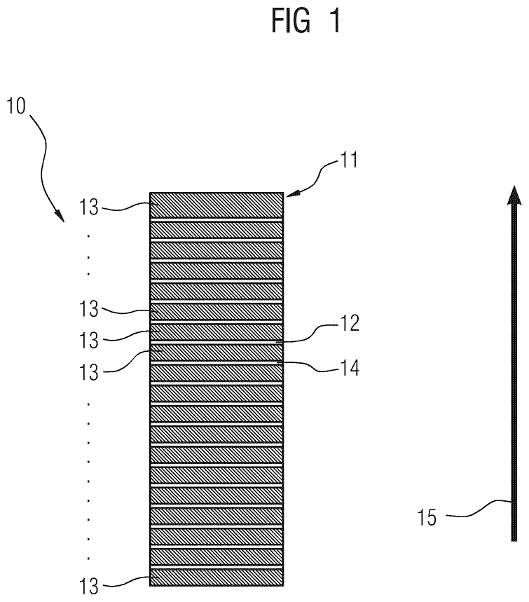

FIG. 1 shows a schematic diagram of a first variant of an inventive absorber mask 10 as a cross section in the width direction. The diagram of the absorber mask 10 is not true to scale.

The inventive absorber mask 10 for shaping a plurality of therapeutic x-rays, especially for radiation therapy of a patient P, has a stack of slices 11. The stack of slices 11 has a number of x-ray radiation-absorbing slices 13. The number of x-ray radiation-absorbing slices 13 each have at least one planar layer. Neighboring layers of the stack of slices 11 are applied onto one another in a planar manner in the stack direction via an additive manufacturing method. The arrow indicates the stack direction 15.

FIG. 1 shows the first variant of the inventive absorber mask 10, wherein at least one x-ray radiation-transparent slice 12, 14, but no x-ray radiation-absorbing strip extending in the width direction, is arranged between the number of x-ray radiation-absorbing slices 13. Preferably x-ray radiation-transparent slices 12, 14 are arranged between all of the number of x-ray radiation-absorbing slices 13.

The at least one x-ray radiation-transparent slice 12, 14 can be embodied from a first material composition, wherein the first material composition has hollow glass spheres and/or an aerogel. The number of x-ray radiation-absorbing slices 13 can be embodied from a second material composition, wherein the second material composition features lead, tantalum, tungsten, rhenium, osmium, iridium, bismuth, platinum, thallium, mercury or gold. The first material composition can have a plastic matrix and the second material composition can have the same plastic matrix. As an alternative the first material composition and the second material composition can have different plastic matrices.

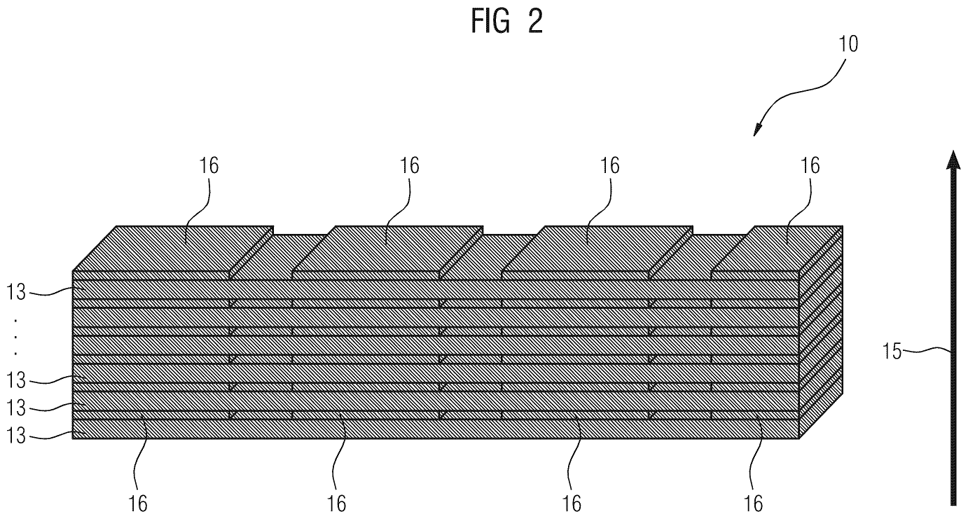

FIG. 2 shows a perspective view of a second variant of an inventive absorber mask 10. By comparison with FIG. 1, the diagram is essentially rotated through 90°, so that the width direction goes into the image plane. The diagram of the absorber mask 10 is not true to scale and especially not representative in respect of the number of strips on the length of the absorber mask.

FIG. 2 shows the second variant of the inventive absorber mask 10, wherein at least one x-ray radiation-absorbing strip 16 extending in the width direction, but no x-ray radiation-transparent slice 12, 14, is arranged between the number of x-ray radiation-absorbing slices 13. Preferably a number of x-ray radiation-absorbing strips 16, i.e. for each slice, are arranged between all of the number of x-ray radiation-absorbing slices 13.

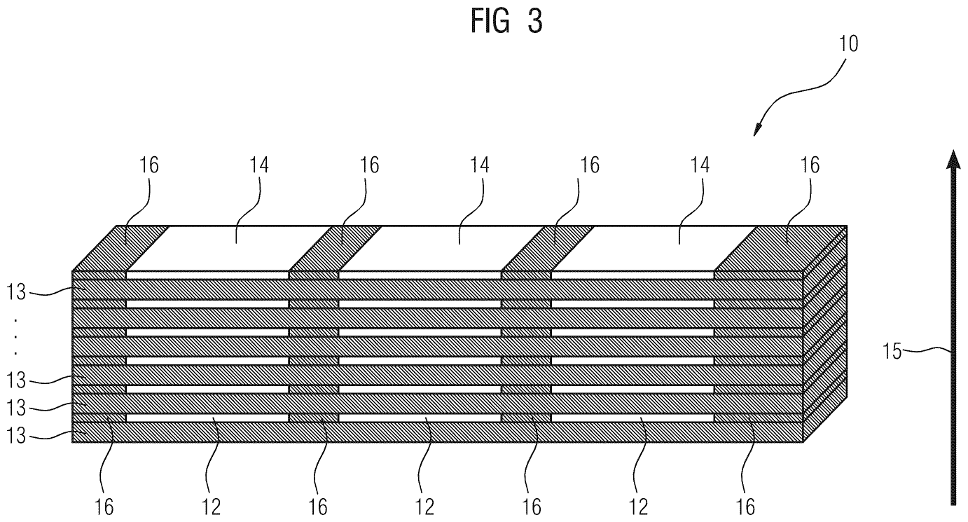

FIG. 3 shows a perspective view of a third variant of an inventive absorber mask 10. By comparison with FIG. 1, the diagram is essentially rotated by 90°, so that the width direction goes into the image plane. The representation of the absorber mask 10 is not true-to-scale and especially not representative in respect of the number of strips on the length of the absorber mask.

FIG. 3 shows the third variant of the inventive absorber mask 10, wherein at least one x-ray radiation-absorbing strip 16 extending in the width direction and at least one x-ray radiation-transparent slice 12, 14 is arranged between the number of x-ray radiation-absorbing slices 13. Preferably a number of x-ray radiation-absorbing strips 16, also for each slice, are arranged between all of the number of x-ray radiation-absorbing slices 13. Preferably x-ray radiation-transparent slices 12, 14 are arranged between all of the number of x-ray radiation-absorbing slices 13.

FIG. 2 and FIG. 3 thus in particular show by comparison that the extent of the strips 16 can vary in the longitudinal extent of the absorber mask 10 and that x-ray radiation-transparent material can be filled in the spaces between the strips or that these are free of material. These spaces, also openings or voids, serve to shape the plurality of therapeutic x-rays. In the case of FIGS. 2 and 3 with the strips, the therapeutic x-rays are rather needle-shaped and in the case of FIG. 1 rather strip-shaped.

FIG. 3 further shows that a thickness of one of the number of x-ray radiation-absorbing slices 13 is greater than a thickness of the x-ray radiation-transparent slice 12, 14 or of the at least one x-ray radiation-absorbing strip 16. The x-ray radiation-absorbing strips 16 of neighboring layers are combined into a regular structure.

One form of embodiment makes provision for a space within a layer between x-ray radiation-absorbing strips 16 to amount to less than or equal to 10 mm, preferably to less than or equal to 1 mm. As an alternative or in addition, a space within a layer between x-ray radiation-absorbing strips can amount to greater than or equal to 0.01 mm, preferably to greater than or equal to 0.1 mm. In particular a ratio within a layer between a length of an x-ray radiation-absorbing strip and a neighboring space can amount to greater than or equal to 1, preferably to greater than or equal to 2. The space between two x-ray radiation-absorbing strips can in particular be greater when x-ray radiation-transparent material is arranged between these x-ray radiation-absorbing strips.

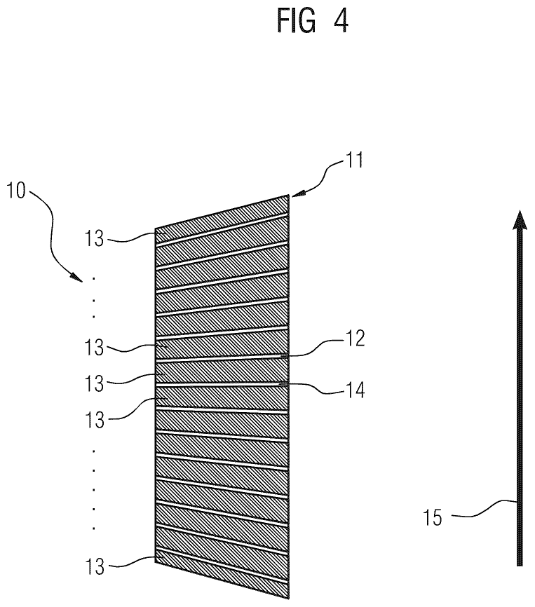

FIG. 4 shows a schematic diagram of a first exemplary embodiment of the first variant of the inventive absorber mask 10 as a section in the width direction. The representation of the absorber mask 10 is not true-to-scale.

The cross section of at least one layer in the width direction is embodied trapezoidal. This makes the absorber mask 10 a focused absorber mask. The absorber mask 10 can especially be focused on the target region in the tissue of the patient P. The trapezoidal shape of the number of x-ray radiation-absorbing slices 13 especially defines a focal point.

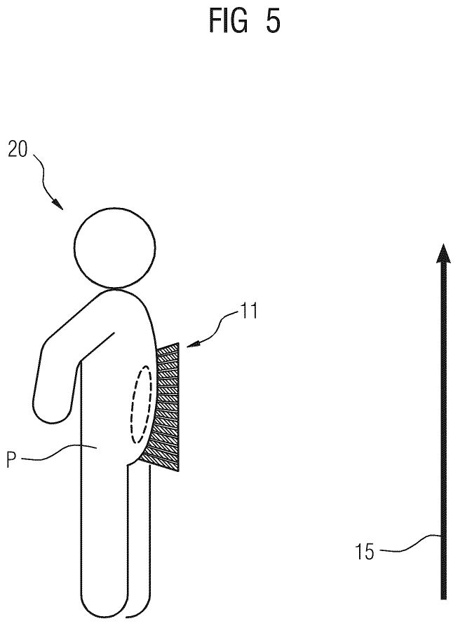

FIG. 5 shows a schematic diagram of a second exemplary embodiment of the first variant of the inventive absorber mask 10 as a cross section in width direction. The diagram of the absorber mask 10 is not true-to-scale.

The stack of slices 11 is produced depending on a contour of the patient P in such a way that one side of the absorber mask 10 essentially has the negative of the contour of the patient P. By comparison with FIG. 4 the upper side of the absorber mask is shaped in such a way as to be an exact fit on the, patient P. Within the patient P the target region is identified by a dashed-line circle. Preferably the focal point of the number of x-ray radiation-absorbing slices 13 lies in the target region of the tissue of the patient P.

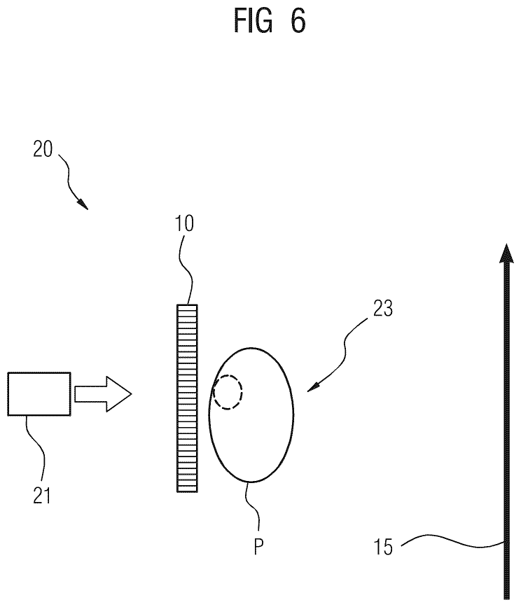

FIG. 6 shows a schematic diagram of an inventive arrangement 20. The diagram is not true-to-scale.

The arrangement 20 for a radiation therapy of a patient has an absorber mask 10, a therapeutic x-ray source 21 and a therapy area 23. The absorber mask 10 is arranged between the therapy area 23 and the therapeutic x-ray source 21 and is aligned in such a way that the stack direction 15 of the stack of slices 11 is essentially at right angles to the x-ray radiation of the therapeutic x-ray source 21. Within the patient P the target region is identified by a dashed-line circle.

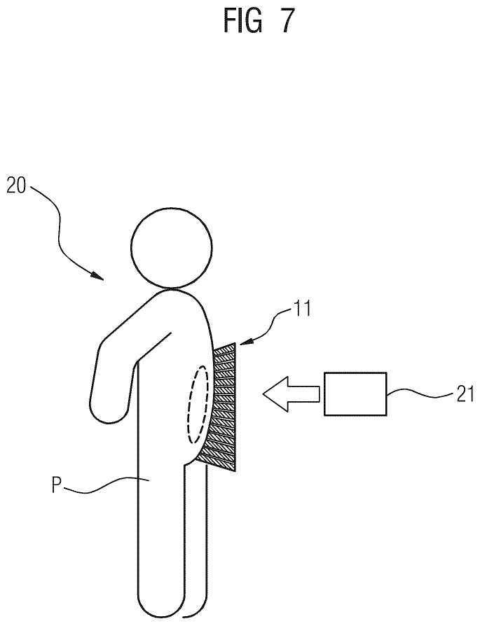

FIG. 7 shows a schematic diagram of a first exemplary embodiment of the inventive arrangement 20. The diagram is not true-to-scale.

The absorber mask 10 is arranged on the contour of the patient. A position of the focal point of the number of x-ray radiation-absorbing slices 13 lies close to the contour of the patient P.

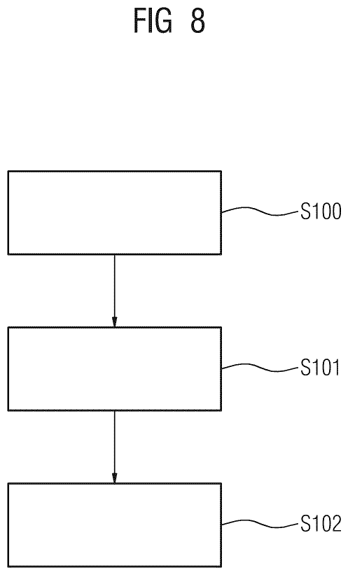

FIG. 8 shows a flow diagram of an inventive method with the steps S100 to S102. The method or individual or all method steps S100 of S102 of the method shown in FIG. 8 can be carried out repeatedly in order to create a stack of slices 11 with a number of x-ray radiation-transparent slices 13, wherein at least one x-ray radiation-transparent slice 12, 14 and/or at least one x-ray radiation-absorbing strip 16 extending in the width direction is arranged between the number of x-ray radiation-absorbing slices 13.

Method steps S100 to S102 identify an application by an additive manufacturing method of layers in a planar manner onto one another in the stack direction to form a stack of slices in such a way that,
the stack of slices 11 has a number of x-ray radiation-absorbing slices 13 and
the stack of slices 11 has at least one x-ray radiation-transparent slice 12, 14 and/or at least one x-ray radiation-absorbing strip 16 extending in the width direction between the number of x-ray radiation-absorbing slices 13. In one or in each of the method steps S100 to S102 a thickness of a layer of the stack of slices 11 can basically be varied in the width direction by the additive manufacturing method, especially to embody a cross section of this layer in the width direction in a trapezoidal shape.

The x-ray radiation-transparent slices 12, 14 can or will be embodied from a first material composition and the number of x-ray radiation-absorbing slices 13 are or will be embodied from a second material composition, wherein the first material composition and the second material composition are embodied differently from one another as regards a proportion of a material. The first material composition can have hollow glass spheres and/or an aerogel. As an alternative or in addition the second material composition can feature lead, tantalum, preferably tungsten, rhenium, osmium, iridium, bismuth, platinum, thallium, mercury or gold. It is conceivable for the first material composition to have a plastic matrix and for the second material composition to have the same plastic matrix.

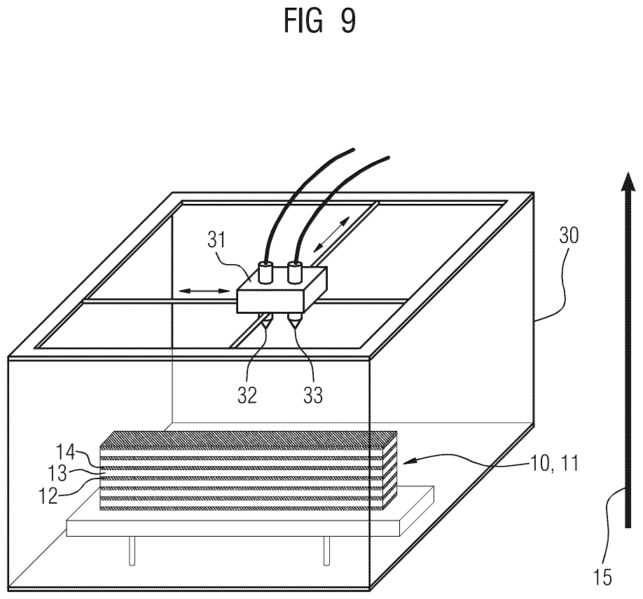

FIG. 9 shows a perspective view of an inventive system 30 for additive manufacturing of an absorber mask 10.

The system 30 has an application unit 31, which is embodied for carrying out an inventive manufacturing method for manufacturing the absorber mask 10. The system 30 can have a number of components, which can be arranged in a vertical configuration. Arranged at the lower part of the system 30 can be a plate, as shown in FIG. 9, which can carry the stack of slices 11 and especially move it vertically. This can make possible an adaptation of the space between the application unit 31 and the stack of slices 11 when layers are applied. Arranged above the stack of slices 11, as shown in FIG. 9 can be a frame structure. This frame structure can be positioned parallel to the plate and be movable in a horizontal plane, which is indicated by a double-ended arrow. The application unit 31 can be mounted on the frame structure. The system 30 of FIG. 8 is especially designed as a “core xy” arrangement, which can make possible efficient additive production.

The application unit 31 of FIG. 9 has two application nozzles 32, 33, which can be used for application of x-ray radiation-transparent material and x-ray radiation-absorbing material to form the slices 12, 13, 14 or the strips 16 of the absorber mask 10. The application unit 31 can be moveable along the frame structure, which can make possible the application of material over the entire surface of the stack of slices lying below it. The application unit 31 can be connected via a number of cables or pipes. These can serve to supply the application unit 31 with materials, energy or control signals. The application nozzles 32, 33 can each have an oval or rectangular cross section.

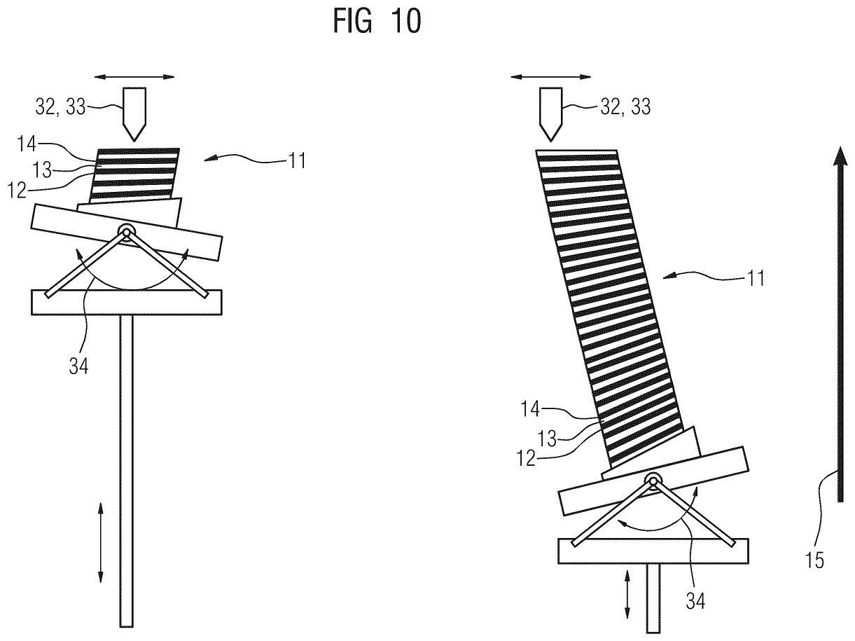

FIG. 10 shows a schematic side view of a first exemplary embodiment of the system. Contained in FIG. 10 are two configurations of the system 30 at two different points in time of the manufacturing of the stack of slices 11.

The system 30 of FIG. 10 has a tilt apparatus 34 below the stack of slices 11. The tilt apparatus 34 is embodied to set the tilt angle of the stack of slices 11 relative to the application nozzles 32, 33. The tilt apparatus 34 is especially embodied to change the tilt angle of the application unit 31 relative to a layer already applied.

In the left-hand configuration the plate on which the stack of slices 11 is applied layer by layer is tilted to the right. The application nozzles 32, 33 are embodied to apply a layer to a layer already produced. In the right-hand configuration the tilt apparatus 34 has adapted the angle of the stack of slices 11 in such a way that the plate is tilted to the left. The tilt apparatus 34 can especially set a different tilt angle for each layer. The tilt angle typically varies gradually from layer to layer in the stack direction 15.

The application nozzles 32, 33 are shown with horizontal arrows, which indicates their lateral movement during the application process. In both configurations the stack of slices 11 shows alternating light and dark slices, which can represent the x-ray radiation-transparent slices 12, 14 and x-ray radiation-absorbing slices 13.

The construction of the system 30 can make the manufacturing of focused absorber masks 10 possible, in that the angle of application can be set by the tilt apparatus 34. This can make possible the production of masks with varying geometries in order to fulfil various requirements of radiation therapy. This form of embodiment is especially advantageous when a thickness of a layer or of each layer of the stack of slices 11 is varied in the width direction by the additive manufacturing method. This especially enables a focused absorber mask to be produced, in which a cross section of a layer or of each layer is embodied in a trapezoidal shape in the width direction.

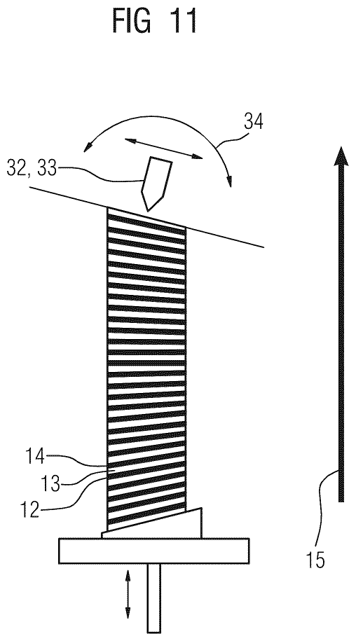

FIG. 11 shows a schematic side view of a second exemplary embodiment of the system 30. The exemplary embodiment of FIG. 11 can be an alternative or an addition to the exemplary embodiment of FIG. 10.

The system 30 of FIG. 11 has a tilt apparatus 34, which is embodied, via the tilt apparatus 34, to change a tilt angle of the application unit 31 relative to a layer already applied. In this exemplary embodiment the tilt apparatus 34 is arranged for example between the frame structure and the application unit 31, so that the application unit 31, especially the two application nozzles 32, 33 are able to be tilted in relation to the stack direction 15. By comparison, the application nozzles 32, 33 of FIG. 10 are not able to be tilted in relation to the stack direction 15, but the plate with the stack of slices 11 is able to be tilted in relation to the stack direction 15.

Independent of the grammatical term usage, individuals with male, female or other gender identities are included within the term.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

Although the present invention has been illustrated in greater detail by the and described preferred exemplary embodiments, despite this the present invention is not restricted by the disclosed examples and other variations can be derived herefrom by the person skilled in the art, without departing from the scope of protection of the present invention.

What is claimed is:

1. An absorber mask for shaping a plurality of therapeutic x-rays, the absorber mask comprising:
a stack of slices including a number of x-ray radiation-absorbing slices, the number of x-ray radiation-absorbing slices each having at least one planar layer, wherein
neighboring layers of the stack of slices are applied onto one another in a planar manner in a stack direction via an additive manufacturing method, and
at least one of at least one x-ray radiation-transparent slice or at least one x-ray radiation-absorbing strip extending in a width direction is arranged between the number of x-ray radiation-absorbing slices.

2. The absorber mask as claimed in claim 1, wherein
a thickness of a x-ray radiation-absorbing slices of the number of x-ray radiation-absorbing slices is greater than a thickness of a x-ray radiation-transparent slice or a thickness of at least one x-ray radiation-absorbing strip.

3. The absorber mask as claimed in claim 1, wherein the stack of slices is produced based on a contour of a patient such that a side of the absorber mask has a negative of the contour of the patient.

4. The absorber mask as claimed in claim 1, wherein the at least one x-ray radiation-transparent slice is embodied from a first material composition, and wherein the first material composition includes at least one of hollow glass spheres or an aerogel.

5. The absorber mask as claimed in claim 4, wherein the number of x-ray radiation-absorbing slices are embodied from a second material composition, and wherein the second material composition includes lead, tantalum, tungsten, rhenium, osmium, iridium, bismuth, platinum, thallium, mercury or gold.

6. The absorber mask as claimed in claim 5, wherein the first material composition has a plastic matrix and the second material composition has a same plastic matrix as the first material composition.

7. The absorber mask as claimed in claim 1, wherein a space within a layer between x-ray radiation-absorbing strips is less than or equal to 10 mm.

8. The absorber mask as claimed in claim 1, wherein a space within a layer between x-ray radiation-absorbing strips is greater than or equal to 0.01 mm.

9. The absorber mask as claimed in claim 1, wherein x-ray radiation-absorbing strips of neighboring layers are composed of a regular structure.

10. The absorber mask as claimed in claim 1, wherein a ratio within a layer between a length of an x-ray radiation-absorbing strip and a neighboring space is greater than or equal to 1.

11. The absorber mask as claimed in claim 1, wherein a cross section of a layer has a trapezoidal shape in a width direction.

12. An arrangement for radiation therapy of a patient, the arrangement comprising:
the absorber mask as claimed in claim 1;
a therapeutic x-ray source; and
a therapy area; wherein
the absorber mask is arranged between the therapy area and the therapeutic x-ray source and is aligned such that the stack direction of the stack of slices is substantially at right angles to x-ray radiation of the therapeutic x-ray source.

13. An arrangement for radiation therapy of a patient, the arrangement comprising:
the absorber mask as claimed in claim 3;
a therapeutic x-ray source; and
a therapy area; wherein
the absorber mask is arranged between the therapy area and the therapeutic x-ray source and is aligned such that the stack direction of the stack of slices is substantially at right angles to x-ray radiation of the therapeutic x-ray source,
the absorber mask is arranged on the contour of the patient, and
a position of a focal point of the number of x-ray radiation-absorbing slices is close to the contour of the patient.

14. A method for manufacturing an absorber mask for shaping a plurality of therapeutic x-rays, the method comprising:
applying, by an additive manufacturing method, layers onto one another in a planer manner and in a stack direction to form a stack of slices such that the stack of slices has a number of x-ray radiation-absorbing slices, and such that the stack of slices has at least one of at least one x-ray radiation-transparent slice or at least one x-ray radiation-absorbing strip extending in a width direction between the number of x-ray radiation-absorbing slices.

15. A system for additive manufacturing of the absorber mask as claimed in claim 1.

16. The absorber mask of claim 1, wherein the plurality of therapeutic x-rays are for radiation therapy of a patient.

17. The absorber mask as claimed in claim 7, wherein the space within the layer between x-ray radiation-absorbing strips is less than or equal to 1 mm.

18. The absorber mask as claimed in claim 8, wherein the space within the layer between x-ray radiation-absorbing strips is greater than or equal 0.1 mm.

19. The absorber mask as claimed in claim 10, wherein the ratio within the layer between the length of the x-ray radiation-absorbing strip and the neighboring space is greater than or equal to 2.

20. A system for additive manufacturing of an absorber mask, the system comprising:
an application unit configured to carry out the method as claimed in claim 14.
